(19) United States
(12) Patent Application Publication
(10) Pub. No.: US 2013/0282082 A1
(43) Pub. Date: Oct. 24, 2013

(54) LIGHT IRRADIATION DEVICE, AND LIGHT IRRADIATION TREATMENT AND PREVENTION DEVICE PROVIDED THEREWITH

(71) Applicants: Kazuhiro Daijo, Osaka (JP); Nobuto Tsujikawa, Osaka (JP); Kazuto Shigehara, Osaka (JP)

(72) Inventors: Kazuhiro Daijo, Osaka (JP); Nobuto Tsujikawa, Osaka (JP); Kazuto Shigehara, Osaka (JP)

(73) Assignee: PANASONIC CORPORATION, Osaka (JP)

(21) Appl. No.: 13/978,739

(22) PCT Filed: Mar. 23, 2012
(86) PCT No.: PCT/JP2012/002020

§ 371 Date: Jul. 9, 2013

(30) Foreign Application Priority Data
Mar. 28, 2011 (JP) 2011-069241

(51) Int. Cl. A61N 5/06

(52) U.S. Cl. CPC A61N 5/0603
USPC 607/92

(57) ABSTRACT

A light irradiation device of the present invention includes: a light source that emits radiant light to an irradiation object; a reflecting member that reflects the radiant light emitted from the light source toward the irradiation object; and an irradiation member that includes at least two irradiation surfaces for transmitting the radiant light reflected from the reflecting member and irradiating the irradiation object with the radiant light. The light source emits the radiant light from one end side of the irradiation surfaces of the irradiation member, and the reflecting member includes: a first reflecting area that reflects the radiant light emitted from the light source toward a first irradiation surface of the irradiation member; a second reflecting area that reflects the radiant light emitted from the light source and indirectly reaches the second reflecting area, toward a second irradiation surface of the irradiation member; and a third reflecting area that guides the radiant light to the second reflecting area. Therefore, the light irradiation device and a light irradiation treatment and prevention device, which can irradiate a plurality of irradiation surfaces with the radiant light using one light source, can be configured.

### TECHNICAL FIELD

The present invention relates to a light irradiation device that can homogeneously irradiate an irradiation object with light and a light irradiation treatment and prevention device provided with the light irradiation device.

### BACKGROUND ART

Conventionally, there have been proposed various light irradiation devices that irradiate the irradiation object with light. For example, there is a light irradiation device including an irradiation member (an irradiation surface) that irradiates the irradiation object, which is uniformly disposed and spread in a planar manner, with light in a planar manner.

An automatic-vending-machine lighting apparatus is proposed as the light irradiation device provided with the irradiation member in response to requests such as save energy, save space, and downsizing (for example, refer to PTL 1).

The automatic-vending-machine lighting apparatus disclosed in PTL 1 includes a planar diffusion panel that is disposed close to a plurality of commercial samples arrayed in vertical and horizontal directions, a planar reflecting sheet that is disposed to face an opposite side (backside) to the commercial sample of the diffusion panel, and a light source that is disposed on the same side-end side of the diffusion panel and reflecting sheet. The reflecting sheet is obliquely disposed with respect to the diffusion panel such that a distance between the reflecting sheet and the diffusion panel is shortened (narrowed) with distance from the light source.

Therefore, in the automatic-vending-machine lighting apparatus, the reflecting sheet reflects the light from the light source disposed on one side-end side toward the whole surface of the diffusion panel. At this point, an optical path length is shortened by shortening the distance from the reflecting sheet to the diffusion panel with distance from the light source, and the automatic-vending-machine lighting apparatus is designed such that illuminance is not lowered at the irradiation object irradiated with the light even if the irradiation object is distant from the light source.

That is, the conventional automatic-vending-machine lighting apparatus is designed such that the optical path length of the light emitted from the light source toward the reflecting sheet is controlled to homogenize the illuminance on the diffusion panel.

There has been devised a light irradiation treatment and prevention device that treats a human or an animal and prevents a disease by the irradiation of the light. It is conceivable that the automatic-vending-machine lighting apparatus in which the illuminance of the diffusion panel is homogenized is applied to the light irradiation treatment and prevention device.

However, the conventional automatic-vending-machine lighting apparatus deals with only the irradiation of the irradiation object from one side. In order to irradiate a region to be treated, for example, in order to simultaneously irradiate a surface and a backside of a hand, it is necessary that the automatic-vending-machine lighting apparatuses be disposed to face both the surfaces of the hand.

In the above configuration, unfortunately the number of components increases, and energy consumption increases. Therefore, there is a strong demand for the light irradiation device and the like that can irradiate the plurality of irradiation surfaces, for example, both the surfaces of the hand using one light source.

### CITATION LIST

### Patent Literature

PTL 1: Unexamined Japanese Patent Publication No. 2009-282725

### SUMMARY OF THE INVENTION

In order to achieve the above object, a light irradiation device of the present invention includes: a light source that emits radiant light to an irradiation object; a reflecting member that reflects the radiant light emitted from the light source toward the irradiation object; and an irradiation member that includes at least two irradiation surfaces for transmitting the radiant light reflected from the reflecting member and irradiating the irradiation object with the radiant light. The light source emits the radiant light from one end side of the irradiation surfaces of the irradiation member, and the reflecting member includes: a first reflecting area that reflects the radiant light emitted from the light source toward a first irradiation surface of the irradiation member; a second reflecting area that reflects the radiant light emitted from the light source and indirectly reaches the second reflecting area toward a second irradiation surface of the irradiation member; and a third reflecting area that guides the radiant light to the second reflecting area.

Therefore, the radiant light reflected by each reflecting area is incident to and transmitted through the irradiation surface corresponding to the irradiation member, and the irradiation object can be irradiated with the radiant light. As a result, using one light source, the irradiation object having the surface and the backside, for example can simultaneously be irradiated with the pieces of radiant light transmitted through the plurality of irradiation surfaces.

### BRIEF DESCRIPTION OF DRAWINGS

FIG. 1A is a graph illustrating a production quantity of a human vascular endothelial growth factor (hVEGF) in each wavelength in a first exemplary embodiment of the present invention.

FIG. 1B is a graph illustrating a production ratio of an inflammatory cytokine in each wavelength in the first exemplary embodiment of the present invention.

FIG. 2 is a control circuit diagram illustrating a light irradiation treatment and prevention device of the first exemplary embodiment of the present invention.

FIG. 3 is an external view illustrating the light irradiation treatment and prevention device of the first exemplary embodiment of the present invention.

FIG. 4 is a cross-sectional view illustrating an optical system of the light irradiation treatment and prevention device of the first exemplary embodiment of the present invention.

FIG. 5 is a graph illustrating spectral characteristics of various band filters including each bandpass filter (a wavelength transmission unit) used in the light irradiation treatment and prevention device of the first exemplary embodiment of the present invention.

FIG. 6 is a block diagram illustrating the light irradiation treatment and prevention device of the first exemplary embodiment of the present invention.

FIG. 7 is a cross-sectional view illustrating a light irradiation device of a light irradiation treatment and prevention device according to a second exemplary embodiment of the present invention.

FIG. 8 is a cross-sectional view illustrating a light irradiation device of a light irradiation treatment and prevention device according to a third exemplary embodiment of the present invention.

### DESCRIPTION OF EMBODIMENTS

Hereinafter, a light irradiation device according to exemplary embodiments of the present invention and a light irradiation treatment and prevention device provided therewith will be described with reference to the drawings. However, the present invention is not limited to the exemplary embodiments.

### First Exemplary Embodiment

A light irradiation device according to a first exemplary embodiment of the present invention and a light irradiation treatment and prevention device provided therewith will be described below with reference to the drawings.

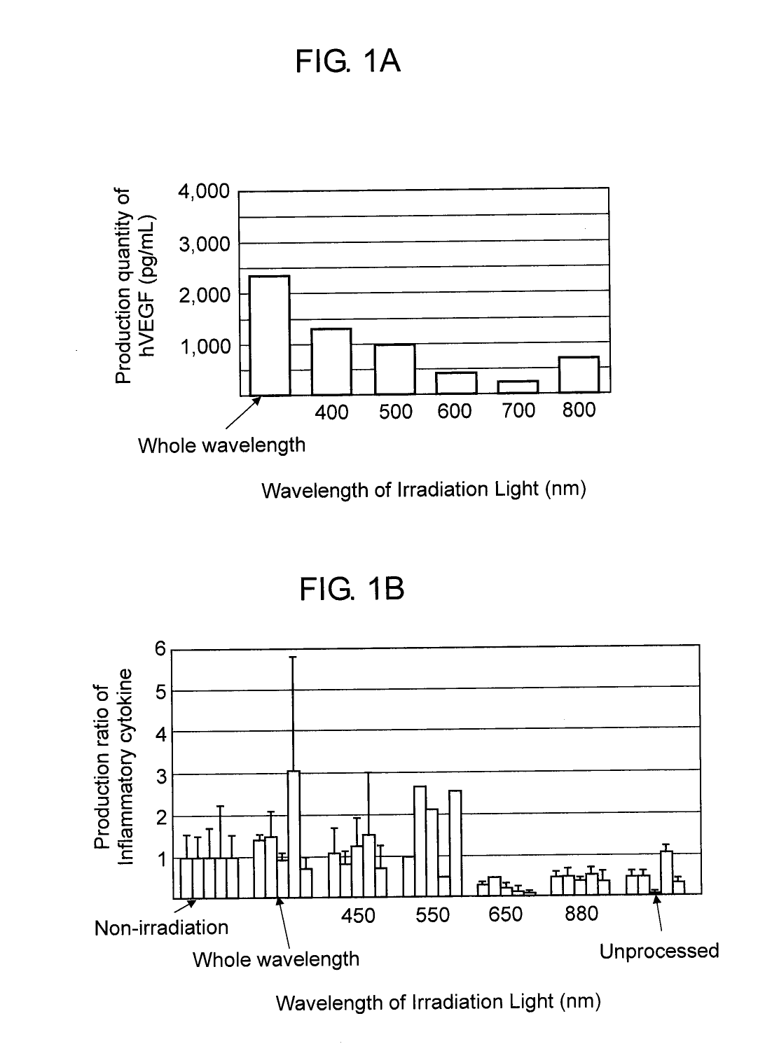

Firstly, a function of an inflammatory cytokine in which production is suppressed by the light irradiation treatment and prevention device of the first exemplary embodiment will be described with reference to FIGS. 1A and 1B.

The inflammatory cytokine is a type of a cytokine that is a general term of soluble proteins playing a role of a wide variety of intercellular signaling in a living body. Particularly, the inflammatory cytokine is a causal factor that causes various symptoms of inflammations in the living body, and the inflammatory cytokine is produced from an activated macrophage or an activated vascular endothelial cell.

Specifically, for example, the inflammatory cytokine is classified into hVEGF (Human Vascular Endothelial Growth Factor), TNFα (tumor necrosis factor-α), IL-1β (interleukin-1β), IFNγ (interferon γ), IL-6 (interleukin-6), and IL-12a (interleukin-12a), which are typical inflammatory cytokines verified by experiments and the like.

The inflammatory cytokine exerts a directed activity as a whole while many types of cytokines form a complicated network in the living body. That is, the inflammatory cytokine raises an excessive disease state of an inflammatory reaction due to a disruption of a balance between the inflammatory cytokine and an anti-inflammatory cytokine that is similarly produced from a blood cell to have an activity suppressing the inflammation.

It is clear from the experiment and the like that IL-4 (interleukin 1α and interleukin-4) that is one of the anti-inflammatory cytokines has no effect to suppress the production of the inflammatory cytokine.

However, the present inventor found that a production quantity of the inflammatory cytokine, for example, the hVEGF is strongly suppressed at a specific wavelength of irradiation light (radiant light) emitted from a discharge tube compared with other wavelengths.

Specifically, as illustrated in FIG. 1A, a xenon discharge tube emitted light to irradiate a human epidermal cell with irradiation light, the irradiation light was dispersed in each predetermined center wavelength with a bandpass filter having a half-value width of 40 nm, and the production quantities of the hVEGF were compared in each center wavelength. As a result, it is found that the production quantity of the hVEGF is minimized between the center wavelength of 600 nm and the center wavelength of 700 nm.

As illustrated in FIG. 1B, similarly, the xenon discharge tube emitted the light to irradiate the human epidermal cell with the irradiation light, the irradiation light was dispersed in each predetermined center wavelength with the bandpass filter having the half-value width of 40 nm, and the production ratios (ratios based on the case of non-irradiation) of the inflammatory cytokine were compared in each center wavelength. As a result, it is found that the production ratio of the inflammatory cytokine becomes the lowest (is strongly suppressed) at the center wavelength of 650 nm.

In FIG. 1B, the production ratio of the inflammatory cytokine is illustrated in each wavelength of the irradiation light based on the production quantity (“1”) of the inflammatory cytokine in the case that the human epidermal cell is not irradiated with the irradiation light. In FIG. 1B, the results of the production ratios of the inflammatory cytokines are illustrated in each wavelength (for example, 450 nm or 550 nm) in the order of the TNFα, the IL-1β, the IFNγ, the IL-6, and the IL-12a from the left.

From the above result, an affected part is irradiated with the irradiation light having the proper wavelength to suppress the production of the inflammatory cytokine, which allows the inflammatory disease to be treated with a new mechanism.

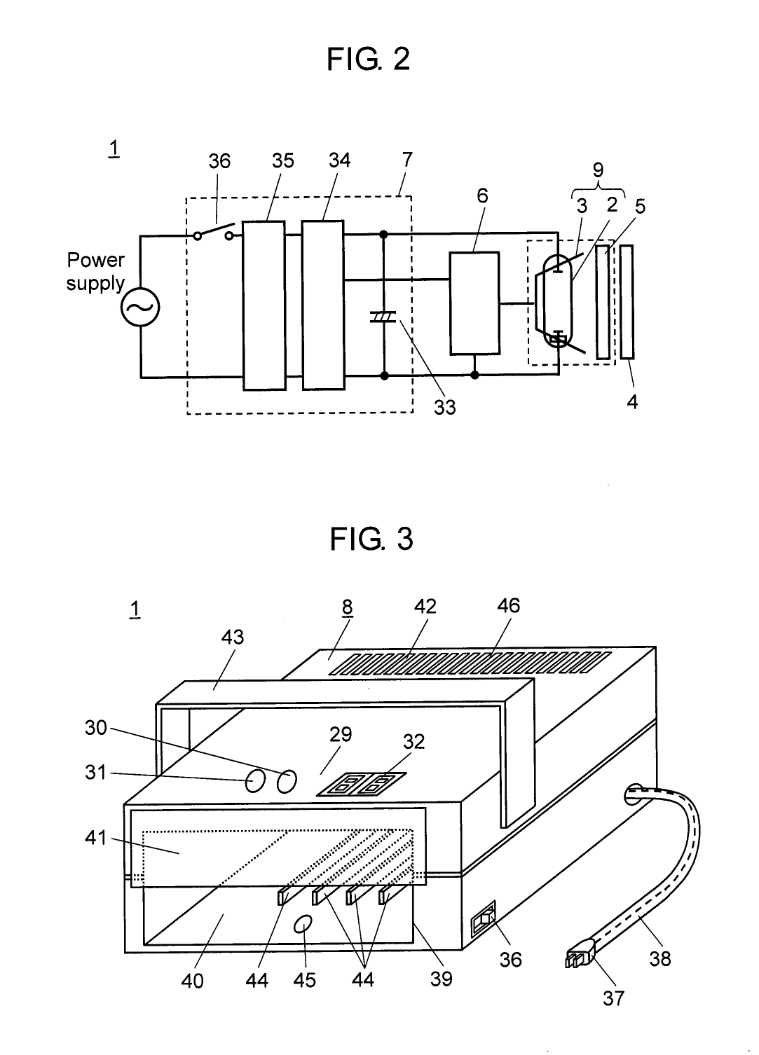

The light irradiation device of the first exemplary embodiment of the present invention and the light irradiation treatment and prevention device provided therewith will be described below with reference to FIGS. 2 to 6.

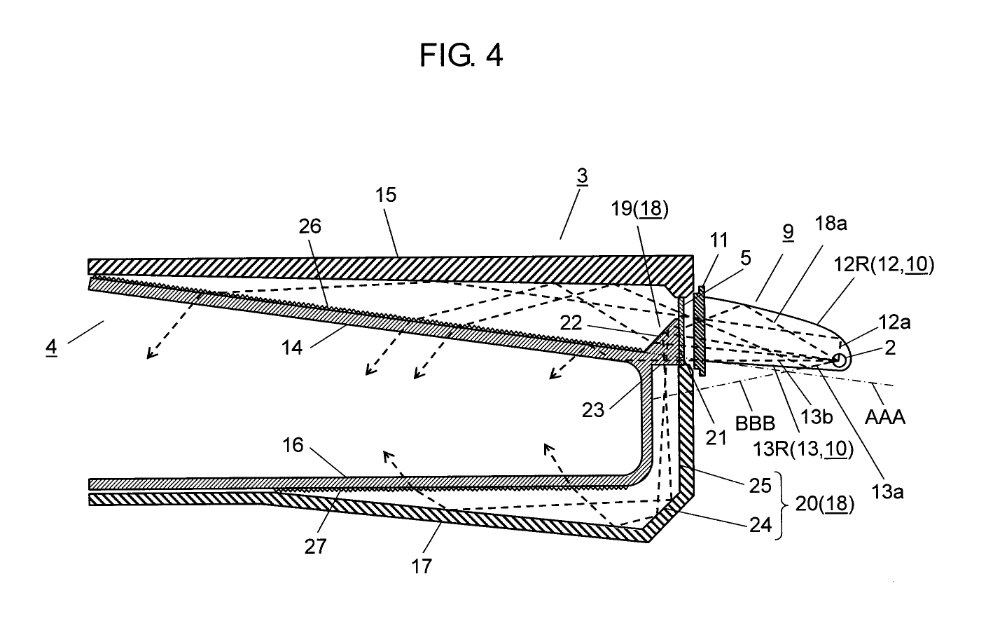

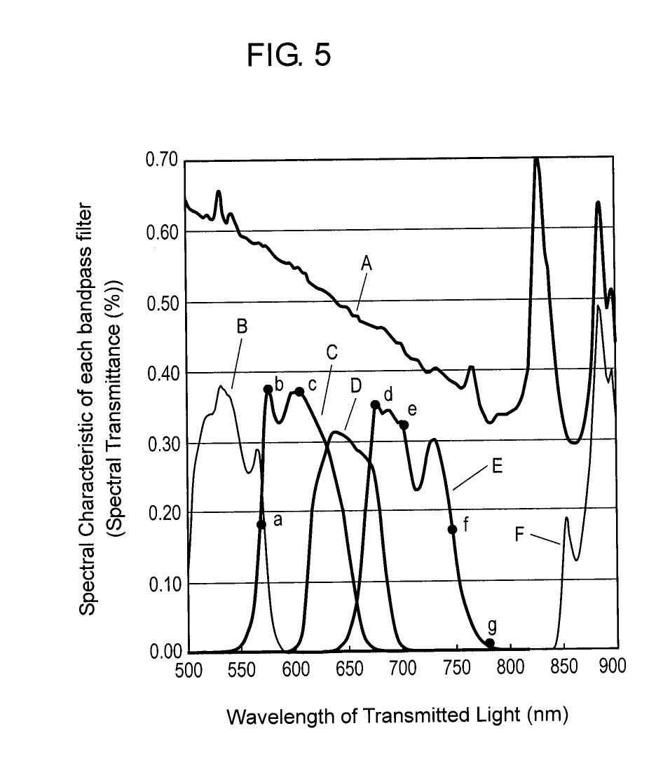

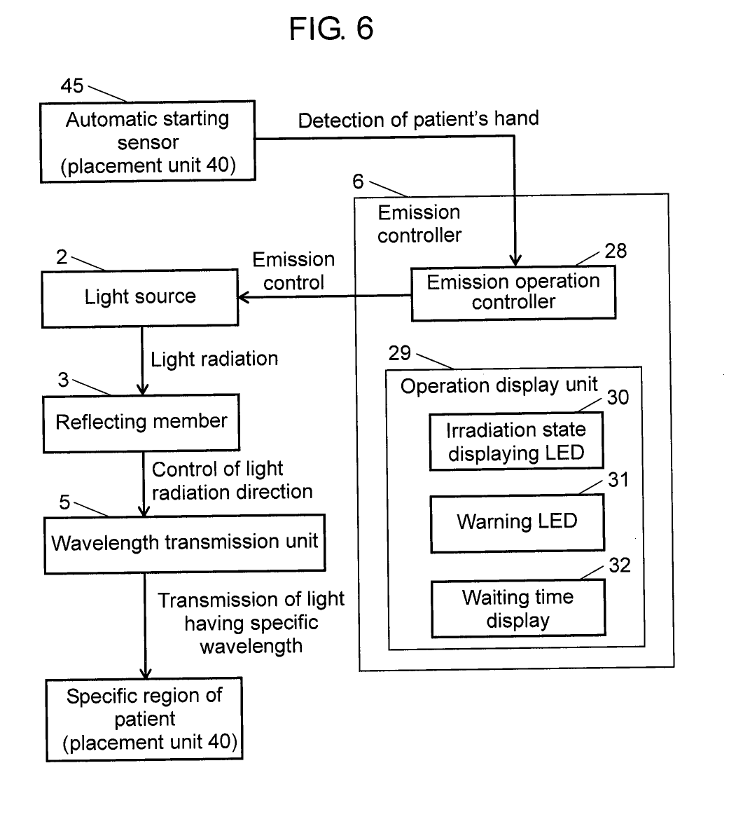

FIG. 2 is a control circuit diagram of a light irradiation treatment and prevention device of the first exemplary embodiment of the present invention. FIG. 3 is an external view illustrating the light irradiation treatment and prevention device of the first exemplary embodiment of the present invention. FIG. 4 is a cross-sectional view illustrating an optical system of the light irradiation treatment and prevention device of the first exemplary embodiment of the present invention. FIG. 5 is a graph illustrating spectral characteristics of the radiant light of a bandpass filter (the wavelength transmission unit) used in the light irradiation treatment and prevention device of the first exemplary embodiment of the present invention. FIG. 6 is a block diagram illustrating the light irradiation treatment and prevention device of the first exemplary embodiment of the present invention.

The light irradiation device, which is used to treat a treated person who prevents the disease of the inflammatory disease or receives a prevention treatment to reduce the symptom of the inflammatory disease or a treated person (a patient) who receives the treatment of the inflammatory disease by suppressing the inflammatory disease, is mainly described as an example of light irradiation treatment and prevention device 1 of the first exemplary embodiment.

As illustrated in FIG. 2, light irradiation treatment and prevention device 1 of the first exemplary embodiment includes light source unit 9 that includes at least light source 2 and Fresnel lens 11 (FIG. 4) to partially constitute reflecting member 3, wavelength transmission unit 5, irradiation member 4, emission controller 6, power-supply unit 7, and device body 8 (FIG. 3). Light source 2 emits the radiant light toward an irradiation object (in FIG. 2, a right side of an irradiation member). Reflecting member 3 includes a reflecting unit and a reflecting area, reflects the radiant light emitted from light source 2 toward the irradiation object. Irradiation member 4 transmits the radiant light reflected from reflecting member 3 to irradiate the irradiation object with the radiant light. Wavelength transmission unit 5 transmits the radiant light having a specific wavelength range in the radiant light emitted from light source 2. Emission controller 6 controls emission of the light source 2, and power-supply unit 7 supplies electricity to light source 2 and emission controller 6.

As illustrated in FIG. 3, device body 8 has a structure in which a region where a user wants to prevent or an affected region (a specific region) can be irradiated with the radiant light transmitted through wavelength transmission unit 5 while light source 2, reflecting member 3, irradiation member 4, wavelength transmission unit 5, emission controller 6, and power-supply unit 7 are accommodated in a rear-side portion (in FIG. 3, a back side). At this point, the user inserts the specific region through opening 39 at a front side in device body 8.

An operation that irradiates the region where the user wants to prevent or the affected region (the specific region) with the radiant light by controlling the emission of light source 2 of light irradiation treatment and prevention device 1 of the first exemplary embodiment will be described in detail below with reference to FIGS. 2, 3, and 6.

As illustrated in FIGS. 2 and 6, emission controller 6 includes emission operation controller 28 and operation display unit 29. Emission operation controller 28 receives a setting of an emission condition of light source 2, and has a function of dealing with a self-diagnosis and a self-diagnosis result. Operation display unit 29 displays an operating state of emission operation controller 28.

That is, emission operation controller 28 controls the emission of light source 2 using the following emission pattern. For example, light source 2 emits flash light once or a plurality of times. In the case that light source 2 emits the flash light a plurality of times, radiation energy radiated from light source 2 may be controlled less than or equal to predetermined radiation energy. Additionally, light source 2 is controlled so as to emit the light at predetermined intervals.

As illustrated in FIGS. 3 and 6, operation display unit 29 includes irradiation state displaying LED 30, warning LED 31, and waiting time display (for example, 7-segment display) 32. For example, irradiation state displaying LED 30 turns green to display a state in which light source 2 can emit the light, when charge of light source 2 is completed to prepare the irradiation of the irradiation light, or when a waiting time (for example, 30 seconds) elapses since the previous irradiation. For example, warning LED 31 turns red and informs the user of generation of a breakdown of light irradiation treatment and prevention device 1 to issue a warning to the user, when power-supply unit 7 becomes abnormal (such as a voltage abnormality and a charge abnormality), or when a temperature in device body 8 is raised. During 30 seconds after the one-time irradiation, in order to not start the next irradiation, warning LED 31 turns on to issue a warming to the user. For example, waiting time display 32 displays the state in which light source 2 can emit the light by “00”, and displays the waiting time until the next state in which light source 2 can emit the light by counting the waiting time until the state in which light source 2 can emit the light to “00” from a numerical value except “00”. Waiting time display 32 also acts as a breakdown display that displays, when the breakdown is generated, an identification number corresponding to the breakdown.

The configuration of the LED or the 7-segment display is described as an example of that of operation display unit 29. However, the configuration of operation display unit 29 is not limited to that of the LED or the 7-segment display. For example, the configuration of operation display unit 29 may be configurations of various lamps and liquid crystal display or a configuration in which the display is performed on a display unit of an external terminal.

Power-supply unit 7 illustrated in FIG. 2 includes electric accumulator 33, charging circuit 34, power unit 35, and power switch 36 (see FIG. 3) that switches between turn-on and -off of power unit 35. Power-supply unit 7 is used as a power supply for emission controller 6.

For example, electric accumulator 33 has an electric capacity necessary for the emission of light source 2, and is configured by a main capacitor connected in parallel with light source 2, and accumulates the emission energy of light source 2. Charging circuit 34 charges electric accumulator 33 using the electricity supplied through power unit 35. For example, power unit 35 includes plug 37 (see FIG. 3) that is connected to a receptacle (an electric outlet) to receive the supply of the electricity and power cable 38 (see FIG. 3), and power unit 35 supplies the electricity to electric accumulator 33. Power unit 35 may be configured to include a battery or a rechargeable battery. Therefore, portability of the light irradiation device is improved.

Device body 8 illustrated in FIG. 3 constitutes a casing. The casing includes at least one opening, and is formed into, for example, a substantially rectangular solid shape, and light source 2, reflecting member 3, irradiation member 4, wavelength transmission unit 5, emission controller 6, and power-supply unit 7 are incorporated in the casing. Device body 8 includes placement unit 40, leaky light preventing unit 41, cooling unit 42, and handle 43 that is gripped to carry device body 8.

Placement unit 40 is a stage in and on which the user inserts and places a user's hand through opening 39 formed in one of surfaces of device body 8 (hereinafter referred to as a “front surface”) in order to irradiate the hand with the radiant light having the wavelength in the specific range. Placement unit 40 includes guide 44 that positions the user's hand inserted through opening 39 and automatic starting sensor 45 that detects a state in which the user's hand is disposed at a predetermined position to be able to be irradiated with the radiant light. A plurality of guides 44 are disposed in parallel with an interval between which each finger of the hand can be inserted. For example, automatic starting sensor 45 is configured by an infrared LED and an infrared phototransistor. Automatic starting sensor 45 detects the insertion of the hand, when the infrared phototransistor does not receive the light emitted from the infrared LED for a predetermined time (for example, at least 2 seconds) because the light is interrupted. The next emission of light source 2 is not started until the infrared phototransistor detects the light emitted from the infrared LED again since the emission of light source 2 is ended.

Leaky light preventing unit 41 is disposed in opening 39 such that an upper half of opening 39 is closed by a light blocking cover preventing the leakage of the irradiation light emitted in placement unit 40 through opening 39 while a space through which the user's hand can be inserted is left. For example, leaky light preventing unit 41 is configured by a neutral density filter (an ND filter) or a shield plate.

Cooling unit 42 cools the inside of device body 8, which becomes a high temperature by light source 2 that is a heat source, using a cooling fan (not illustrated), and exhausts high-temperature air from exhaust port 46 provided in a rear portion (a rear end in an upper surface) of device body 8. For example, cooling unit 42 is configured by an air-cooled system or a water-cooled system according to an application or a cooling capacity.

Light source unit 9 accommodated in device body 8 will be described below.

In this case, the irradiation object, for example, the hand is inserted in device body 8 between first irradiation surface 14 and a second irradiation surface which are described later.

In light source unit 9, as illustrated in the sectional side view of FIG. 4, light source 2, reflector 10 that is a part of reflecting member 3 and constitutes a reflecting unit, and Fresnel lens 11 are integrally configured. Reflector 10 reflects the radiant light emitted from light source 2 toward one direction (the left side in FIG. 4). Fresnel lens 11 is provided in the opening of reflector 10, and matches an incident angle of the light incident toward wavelength transmission unit 5 provided on an opposite side to light source 2 with respect to Fresnel lens 11.

Hereinafter, reflector 10 constituting a part of reflecting member 3 and a remaining portion except a part of reflecting member 3 including first reflecting area 15 and second reflecting area 17 is simply referred to as reflecting member 3.

At this point, as illustrated in FIG. 4, light source 2 of light source unit 9 is disposed on an opposite side (the lower side in FIG. 4) to an irradiation object side of irradiation member 4 with respect to a surface corresponding to a tangent AAA, in which the irradiation surface formed into the substantially planar shape (including the planar shape) is extended, such that one end side (the right side in FIG. 4) of irradiation member 4 is irradiated with the radiant (irradiation) light output from Fresnel lens 11. However, as in the present exemplary embodiment, the case that partially direct incident of light to the irradiation surface of irradiation member 4 is negligible or needs not to be considered when light source 2 is disposed at the position illustrated in FIG. 4 is interpreted as a meaning that “light source 2 is disposed on the irradiation object side with respect to the tangent AAA of the irradiation surface of irradiation member 4” in the description of the present invention. That is, at the position where radiant light 13b illustrated in FIG. 4 that is partially emitted from light source 2 is not directly incident to the irradiation object but totally reflected from the irradiation surface even if radiant light 13b is directly incident to the irradiation surface of irradiation member 4, it is assumed that light source 2 is disposed on the irradiation object side with respect to the tangent AAA even if light source 2 is disposed on the opposite side (the upper side of the tangent AAA in FIG. 4) to the irradiation object side with respect to the tangent AAA. The description thereof will be given below.

On the other hand, in the case that light source 2 is disposed at the position where radiant light emitted from light source 2 is not totally reflected but the irradiation object is directly irradiated with the radiant light, that “light source 2 is disposed on the opposite side to the irradiation object side with respect to the tangent AAA of the irradiation surface of irradiation member 4” is expressed, and the description thereof will be given.

For example, light source 2 is configured by the (flash) discharge tube such as the xenon discharge tube and the halogen discharge tube, and the region to be prevented or the diseased region of the user's living body is irradiated with the radiant light having the wavelength at which the production of the inflammatory cytokine is suppressed. In the first exemplary embodiment, the xenon discharge tube is used by way of example.

Reflector 10 constituting the reflecting unit of reflecting member 3 includes first reflecting plate 12R constituting first reflecting unit 12 and second reflecting plate 13R constituting second reflecting unit 13. In the radiant light emitted from light source 2, for example, first reflecting plate 12R reflects radiant light 12a, which travels onto the opposite side (the inside of the device) to the irradiation object side of irradiation member 4 with respect to the tangent AAA (the surface in which the irradiation surface of irradiation member 4 is extended) of irradiation member 4 (the irradiation surface), toward irradiation member 4. On the other hand, for example, the second reflecting plate 13R reflects radiant light 13a traveling onto the irradiation object side toward first reflecting plate 12R and reflecting member 3.

As illustrated in FIG. 4, specifically, the reflecting surface of first reflecting plate 12R is disposed so as to be opposed to irradiation member 4, and the reflecting surface of second reflecting plate 13R is disposed so as not to be opposed to irradiation member 4 but to be opposed to the reflecting surface of first reflecting plate 12R. Accordingly, for example, radiant light 13a (corresponding to line BBB in FIG. 4) traveling onto the irradiation object side from light source 2 is reflected by the reflecting surface of second reflecting plate 13R, transmitted through Fresnel lens 11 and wavelength transmission unit 5, reflected by first reflecting area 15 of reflecting member 3, and is incident to irradiation member 4.

For example, Fresnel lens 11 is provided in the case that a filter having an incident angle dependence property is used in wavelength transmission unit 5. At this point, Fresnel lens 11 is provided such that the incident angle of the light from light source 2 falls within an allowable range of wavelength transmission unit 5 used. Fresnel lens 11 may be eliminated in the case that a color glass filter having no incident angle dependence property is used as wavelength transmission unit 5.

Reflecting member 3 is configured to include reflector 10 constituting the reflecting unit of light source unit 9, and reflecting member 3 controls the irradiation range of the radiant light emitted in substantially all the directions (including all the directions) from light source 2 such that the region to be prevented or the diseased region (the specific region) is irradiated with the radiant light transmitted through wavelength transmission unit 5. Reflecting member 3 includes first reflecting area 15 that reflects the radiant light emitted from light source 2 toward first irradiation surface 14 of irradiation member 4, second reflecting area 17 that reflects the light indirectly reaching second reflecting area 17 from light source 2 toward second irradiation surface 16 of irradiation member 4, and third reflecting area 18 that guides the radiant light to reach second reflecting area 17.

At this point, as illustrated in FIG. 4, first reflecting area 15 of reflecting member 3 is disposed to face first irradiation surface 14 of irradiation member 4. A distance between first reflecting area 15 of reflecting member 3 and first irradiation surface 14 of irradiation member 4 is formed so as to become narrower with distance from light source 2, and first reflecting area 15 and first irradiation surface 14 are disposed such that an optical path length between light source 2 and first irradiation surface 14 of irradiation member 4 is shortened. Specifically, in the first exemplary embodiment, as illustrated in FIG. 4, for example, first reflecting area 15 of reflecting member 3 is provided in a horizontal direction, and first irradiation surface 14 of irradiation member 4 is obliquely provided such that a distance from first reflecting area 15 of reflecting member 3 is shortened (narrowed) from the side of light source 2 toward the opposite side to light source 2. First reflecting area 15 of reflecting member 3 is formed so as to be substantially continuously (including continuously) connected to first reflecting plate 12R of light source unit 9 constituting the reflecting unit of a part of reflecting member 3, and first reflecting area 15 is provided to the position on the opposite side of light source 2 with respect to first irradiation surface 14.

On the other hand, second reflecting area 17 of reflecting member 3 is disposed on the opposite side of first reflecting area 15 with first irradiation surface 14 and second irradiation surface 16 of irradiation member 4 interposed therebetween. A distance between second reflecting area 17 of reflecting member 3 and second irradiation surface 16 of irradiation member 4 is formed so as to become narrower with distance from light source 2, and second reflecting area 17 and second irradiation surface 16 are disposed such that an optical path length between light source 2 and second irradiation surface 16 of irradiation member 4 is shortened. Specifically, in the first exemplary embodiment, as illustrated in FIG. 4, for example, second irradiation surface 16 of irradiation member 4 is provided in the horizontal direction, and second reflecting area 17 of reflecting member 3 is obliquely provided such that a distance from second irradiation surface 16 of irradiation member 4 is shortened (narrowed) from the side of light source 2 toward the opposite side to light source 2. The obliquity of second reflecting area 17 of reflecting member 3 is formed in the middle of first irradiation surface 14 of irradiation member 4. However, the obliquity is not limited to the middle of first irradiation surface 14. That is, in the light irradiation device of the first exemplary embodiment, it is considered that the surface and backside of the hand are irradiated with the radiant light. Therefore, because the production quantity of the inflammatory cytokine is small in a palm, the palm is not irradiated with the radiant light.

Third reflecting area 18 of reflecting member 3 includes reflecting unit 20. Reflecting unit 20 reflects the radiant light, which is reflected or refracted by prism 19, toward second reflecting area 17 of reflecting member 3. Prism 19 is disposed on part of the optical path of the radiant light that is transmitted through Fresnel lens 11 of light source unit 9 to travel toward first reflecting area 15, and prism 19 is formed in irradiation member 4. In order to reflect the radiant light reflected by the prism 19 toward second reflecting area 17, reflecting unit 20 includes reflecting surface 24 that is substantially parallel (including parallel) to reflecting surface 22 of prism 19 and guide surface 25 that reflects and guides the radiant light from prism 19 to second reflecting area 17 which will be described later.

In third reflecting area 18 of reflecting member 3, for example, radiant light 18a emitted from light source 2 toward first reflecting area 15 is guided to second reflecting area 17 by prism 19 that partially blocks the optical path of radiant light 18a. Prism 19 is disposed so as to cover a half of the side of first irradiation surface 14 of the radiant light transmitted through Fresnel lens 11 of light source unit 9. Therefore, prism 19 distributes the radiant light emitted from light source 2 into first reflecting area 15 and second reflecting area 17 of reflecting member 3.

For example, prism 19 is formed into a right-angled triangular prism shape, and includes incident surface 21, reflecting surface 22, and transmission surface 23. Incident surface 21 of prism 19 is configured by a surface orthogonal to the incidence of the radiant light transmitted through Fresnel lens 11. Reflecting surface 22 of prism 19 is configured by an oblique surface that partially reflects the radiant light incident from incident surface 21 toward second reflecting area 17 through third reflecting area 18. Transmission surface 23 of prism 19 is configured by a surface, which is orthogonal to the radiant light and transmits the radiant light reflected by reflecting surface 22 of prism 19 without substantially refracting (including refracting) the radiant light. Incident surface 21 and transmission surface 23 of prism 19 are orthogonal to each other, and reflecting surface 22 of prism 19 is obliquely formed by 45 degrees angle, for example, with respect to incident surface 21 and transmission surface 23.

Irradiation member 4 includes first irradiation surface 14 and second irradiation surface 16, which are disposed to face each other. At this point, as illustrated in FIG. 4, a plurality of micro prisms 26 and 27 are formed on the incident surface sides of first irradiation surface 14 and second irradiation surface 16 of irradiation member 4 such that a transmission angle (an angle from the surface orthogonal to the irradiation surface) of the radiant light transmitted through irradiation member 4 is refracted at an angle smaller than the incident angle of the radiant light incident to irradiation member 4. Therefore, the radiant light is refracted by the surfaces (the surfaces that do not face the irradiation object) on the opposite side to the irradiation object side in first irradiation surface 14 and second irradiation surface 16 of irradiation member 4 at an angle smaller than the incident angle, and the irradiation object is irradiated with the radiant light.

Wavelength transmission unit 5 is disposed on the optical path of the radiant light emitted from light source 2 toward irradiation member 4 as needed basis. In the first exemplary embodiment, wavelength transmission unit 5 is disposed on the side of light source 2 with respect to incident surface 21 of prism 19 of third reflecting area 18 of reflecting member 3. Wavelength transmission unit 5 is configured by an optical filter that transmits only the radiant light, which is emitted from light source 2 and has at least one specific wavelength range or the wavelength in at least one specific range.

A bandpass filter (an interference filter) that selectively transmits only the radiant light having the specific wavelength range (a wavelength band) will be described below as an example of the optical filter of wavelength transmission unit 5 of the first exemplary embodiment with reference to FIG. 5.

Specifically, wavelength transmission unit 5 is the bandpass filter that transmits the radiant light having wavelengths of 566.5 nm to 780 nm.

FIG. 5 is a graph illustrating spectral characteristics of various band filters including each bandpass filter (a wavelength transmission unit) used in the light irradiation treatment and prevention device of the first exemplary embodiment. Hereinafter, bandpass filters having spectral characteristics indicated by solid lines C to E are referred to as bandpass filters C to E, respectively. The solid line A in FIG. 5 indicates the spectral characteristic of the radiant light that is not transmitted through the optical filter.

As illustrated in FIG. 5, a lower limit of the wavelength range of the spectral characteristic (a spectral transmittance) of the radiant light transmitted through each bandpass filter is the wavelength (the wavelength corresponding to an a point on the solid line C) on the short-wavelength side on which the transmittance of the bandpass filter C, which is indicated by the solid line C and has the center wavelength of 600 nm, becomes a half of the maximum transmittance (at the wavelength of 566.5 nm corresponding to a b point on the solid line C). On the other hand, an upper limit of the wavelength range of the spectral characteristic (the spectral transmittance) of the radiant light transmitted through each bandpass filter is the maximum wavelength of 780 nm of visible light.

That is, as illustrated in FIG. 1A, the bandpass filter C having the center wavelength of 600 nm of the radiant light is the optical filter that transmits the wavelength having the effect to suppress the production quantity of the hVEGF. From the result in FIG. 1A, it is considered that the bandpass filter B, which is indicated by the solid line B illustrated in FIG. 5 and has the spectral characteristic of the center wavelength of 500 nm on the short-wavelength side of the bandpass filter C, has the low effect to suppress the production quantity of the hVEGF. Therefore, it is estimated that the wavelength at the a point on the short-wavelength side of the bandpass filter C having the center wavelength of 600 nm is the lower limit having the effect to suppress the inflammatory cytokine.

On the other hand, because near-infrared (greater than the wavelength of 780 nm) radiant light in FIG. 5 has a large thermal influence on the user when the user irradiated with the near-infrared radiant light, the wavelength range of the visible light (less than or equal to the wavelength of 780 nm) including the near-infrared light is set to the upper limit of the radiant light transmitted through wavelength transmission unit 5.

Preferably, the optical filter that transmits the radiant light having wavelength ranges of 566.5 nm to 746 nm is used as wavelength transmission unit 5. That is, the upper limit (less than or equal to 746 nm) of the wavelength range is the wavelength (the wavelength corresponding to an f point on the solid line E) on the long-wavelength side on which the transmittance of the bandpass filter E, which is indicated by the solid line E in FIG. 5 and has the spectral characteristic of the center wavelength of 700 nm, becomes a half of the maximum transmittance at the wavelength of 676.5 nm corresponding to a d point on the solid line E.

That is, as illustrated in FIG. 1A, the bandpass filter E having the center wavelength of 700 nm of the radiant light is the optical filter that transmits the wavelength having the effect to suppress the production quantity of the hVEGF. From the result in FIG. 1A, it is considered that the bandpass filter F, which is indicated by the solid line F in FIG. 5 and has the spectral characteristic of the center wavelength of 880 nm on the long-wavelength side of the bandpass filter E, has the low effect to suppress the production quantity of the hVEGF. Therefore, it is estimated that the wavelength at the f point on the long-wavelength side of the bandpass filter E having the center wavelength of 700 nm is the value having the effect to suppress the inflammatory cytokine.

On the other hand, the wavelength of 780 nm (the wavelength corresponding to a g point on the solid line E) that is the upper limit of the bandpass filter E is also located on the short-wavelength side of the wavelength of the radiant light transmitted through the bandpass filter F having the center wavelength of 880 nm. Therefore, the effect to suppress the inflammatory cytokine by the irradiation of the radiant light having the wavelength of 780 nm is not denied, but it can be evaluated that the wavelength of 780 nm has the effect to suppress the inflammatory cytokine.

More preferably, the optical filter that transmits the radiant light having wavelength ranges of 600 nm to 700 nm is used as wavelength transmission unit 5.

As illustrated in FIG. 1A, the bandpass filter C having the center wavelength of 600 nm (the wavelength corresponding to a c point on the solid line C), which is the optical filter transmitting the radiant light having the wavelength that can suppress the production quantity of the hVEGF, and the bandpass filter E having the center wavelength of 700 nm (the wavelength corresponding to an e point on the solid line E) are used while combined, for example. This enables the production of the inflammatory cytokine to be suppressed at a high efficiency.

As illustrated in FIG. 1B, the wavelength range of the radiant light transmitted through the bandpass filter D, which is indicated by the solid line D in FIG. 5 and has the spectral characteristic of the center wavelength of 650 nm having the effect to suppress the production of the inflammatory cytokine, is located between the bandpass filter C having the center wavelength of 600 nm and the bandpass filter E having the center wavelength of 700 nm. That is, the wavelength ranges of the bandpass filter C and the bandpass filter E overlap the wavelength range of the bandpass filter D having the spectral characteristic of the center wavelength of 650 nm. For this reason, it is considered that the radiant light having the wavelength transmitted through the bandpass filter C having the center wavelength of 600 nm and the bandpass filter E having the center wavelength of 700 nm has the effect to suppress the production of the inflammatory cytokine.

An operation of light irradiation treatment and prevention device 1 of the first exemplary embodiment of the present invention will specifically be described below.

Firstly, power switch 36 of device body 8 illustrated in FIG. 3 is turned on to check whether irradiation state displaying LED 30 turns green. After the check, the user's hand is inserted through opening 39 of device body 8, and each finger is inserted in guide 44 to dispose the hand at a predetermined position of placement unit 40. Therefore, the user's hand is fixed between first irradiation surface 14 and second irradiation surface 16 of irradiation member 4 illustrated in FIG. 4.

When the user's hand is inserted in the guide 44 to fit at the predetermined position, automatic starting sensor 45 senses the user's hand, emission controller 6 causes light source 2 to emit the light to irradiate the user's hand with the radiant light.

At this point, in the radiant light emitted from light source 2 illustrated in FIG. 4, radiant light 12a (main incident light) that travels toward the opposite side to the irradiation object side from the tangent AAA of first irradiation surface 14 of irradiation member 4 is reflected by first reflecting plate 12R of reflector 10 or radiant light 12a is directly transmitted through Fresnel lens 11 toward first reflecting area 15 or irradiation member 4 while not reflected by first reflecting plate 12R. On the other hand, in the radiant light emitted from light source 2, radiant light 13a (scattering light out of the main incident light) that travels toward the irradiation object from the tangent AAA of first irradiation surface 14 of irradiation member 4 is reflected by second reflecting plate 13R of reflector 10 or further reflected by first reflecting plate 12R of reflector 10, and transmitted through Fresnel lens 11 toward first reflecting area 15 or irradiation member 4.

In the radiant light transmitted through Fresnel lens 11, radiant light 18a, which is transmitted through wavelength transmission unit 5, is incident to third reflecting area 18, and reflected toward second reflecting area 17 by reflecting surface 22 of prism 19, is transmitted through prism 19 while not substantially scattered by transmission surface 23, and radiant light 18a is reflected toward second reflecting area 17 by reflecting surface 24 of reflecting unit 20 of third reflecting area 18. Then, radiant light 18a is reflected by second reflecting area 17 of reflecting member 3, and is incident to second irradiation surface 16 of irradiation member 4. Radiant light 18a incident to second irradiation surface 16 is refracted onto the side of light source 2 by prism 27 of second irradiation surface 16 so as not to be leaked to the outside (the opposite side to light source 2), and the irradiation object (for example, the palm) opposed to second irradiation surface 16 is irradiated with radiant light 18a.

On the other hand, remaining pieces of radiant light 12a and 13a transmitted through Fresnel lens 11 is reflected by first reflecting area 15 of reflecting member 3, and first irradiation surface 14 of irradiation member 4 is irradiated with pieces of radiant light 12a and 13a, or first irradiation surface 14 of irradiation member 4 is directly irradiated with pieces of radiant light 12a and 13a. The radiant light with which first irradiation surface 14 of irradiation member 4 is irradiated is refracted onto the side of light source 2 by prism 26 of first irradiation surface 14 of irradiation member 4, and the irradiation object (for example, a back of the hand) opposed to first irradiation surface 14 is irradiated with the radiant light.

In light irradiation treatment and prevention device 1 of the first exemplary embodiment, in the case that third reflecting area 18 of reflecting member 3 is not disposed on the optical path through which part of the radiant light emitted from light source 2 travels, the radiant light is not reflected by reflector 10 (first reflecting plate 12R and second reflecting plate 13R) and reflecting member 3 (first reflecting area 15 and second reflecting area 17), but the irradiation member 4 can be directly irradiated with the radiant light. This is because light source 2 is disposed near the opposite side to the irradiation object side of irradiation member 4 with respect to the tangent AAA of first irradiation surface 14 of irradiation member 4.

However, in the case that the radiant light with which irradiation member 4 is directly irradiated is negligible, in the case that illuminance of the radiant light with which irradiation member 4 is irradiated needs not to be considered, or in the case that the resultant illuminance of the radiant light with which irradiation member 4 is directly irradiated from first irradiation surface 14 is controlled by prism 26 provided in first irradiation surface 14 of irradiation member 4 or third reflecting area 18 of reflecting member 3 like the first exemplary embodiment, as described above, light irradiation treatment and prevention device 1 has the same effect as the case that light source 2 is disposed on the irradiation object side with respect to the tangent AAA of first irradiation surface 14 of irradiation member 4. Therefore, the same interpretation as that “light source 2 is disposed on the irradiation object side with respect to the tangent AAA of first irradiation surface 14 of irradiation member 4” of the first exemplary embodiment is taken.

According to the first exemplary embodiment, light source 2 can be disposed such that the emitted radiant light is not directly incident to first irradiation surface 14 and the second irradiation surface of irradiation member 4. That is, the light, which is emitted from light source 2 and reflected by reflector 10 and reflecting member 3, is incident to first irradiation surface 14 and second irradiation surface 16. At this point, the radiant light incident to irradiation member 4 that is emitted from light source 2 to travel toward the opposite side to the irradiation object side with respect to the tangent AAA of first irradiation surface 14 of irradiation member 4 is reflected by first reflecting plate 12R of reflector 10 or first reflecting area 15 of reflecting member 3, and is incident to first irradiation surface 14 and second irradiation surface 16. On the other hand, the light (the scattering light out of the main incident light) that travels toward the irradiation object side with respect to the tangent AAA of first irradiation surface 14 of irradiation member 4 is reflected toward first reflecting plate 12R of reflector 10 or reflecting member 3 by second reflecting plate 13R of reflector 10, and is incident to first irradiation surface 14 and second irradiation surface 16. For this reason, the optical path length of the scattering light is longer than the optical path length of the main incident light by a roundabout length in which the scattering light is reflected by second reflecting plate 13R of reflector 10. Therefore, even if the scattering light reflected by second reflecting plate 13R is caused to merge with the main incident light by first reflecting plate 12R, the illuminance at the irradiation surface of irradiation member 4 is not locally excessively strengthened. As a result, the radiant light (the main incident light and the scattering light) emitted from light source 2 is completely guided to the irradiation surface of irradiation member 4 by first reflecting plate 12R and second reflecting plate 13R, and reflected by first reflecting area 15 and second reflecting area 17 of reflecting member 3, which allows first irradiation surface 14 and second irradiation surface 16 of irradiation member 4 to be homogeneously irradiated with the radiant light.

According to the first exemplary embodiment, for example, irradiation member 4 includes first irradiation surface 14 and second irradiation surface 16. Part of the radiant light emitted from light source 2 toward first irradiation surface 14 and second irradiation surface 16 of irradiation member 4 is reflected by third reflecting area 18 and indirectly guided to second reflecting area 17, and the radiant light is reflected toward first irradiation surface 14 and second irradiation surface 16 of irradiation member 4 by one of first reflecting area 15 and second reflecting area 17 of reflecting member 3. Therefore, the radiant light reflected by first reflecting area 15 and second reflecting area 17 is incident to and transmitted through first irradiation surface 14 and second irradiation surface 16 of irradiation member 4, and the irradiation object can be irradiated with the radiant light. As a result, for example, using one light source 2, the irradiation object having the surface and the backside can simultaneously be irradiated with the radiant light transmitted through first irradiation surface 14 and second irradiation surface 16.

According to the first exemplary embodiment, in the radiant light that is emitted from light source 2 toward first reflecting area 15, part of the radiant light in which the optical path to first reflecting area 15 is blocked by prism 19 of third reflecting area 18 is guided to second reflecting area 17 and transmitted through second irradiation surface 16, and the irradiation object is irradiated with the radiant light. On the other hand, in the radiant light emitted from light source 2 toward first reflecting area 15, the remaining radiant light that is not blocked by prism 19 is directly transmitted through first irradiation surface 14, and the irradiation object is irradiated with the radiant light. Therefore, the pieces of radiant light that are emitted from light source 2 to travel toward second reflecting area 17 through first reflecting area 15 and third reflecting area 18 are transmitted through first irradiation surface 14 and second irradiation surface 16 corresponding to each reflecting area, and the irradiation object can simultaneously be irradiated with the pieces of radiant light.

According to the first exemplary embodiment, in the radiant light output from irradiation member 4, the radiant light is refracted onto and transmitted through the sides of light source 2 of first irradiation surface 14 and second irradiation surface 16 by prism 26 of first irradiation surface 14 and prism 27 of second irradiation surface 16 of irradiation member 4, and the irradiation object is irradiated with the radiant light. Therefore, the irradiation object can homogeneously be irradiated with the radiant light output from irradiation member 4 such that the radiant light does not spread to the outside of the light irradiation device.

According to the first exemplary embodiment, in the radiant light that is emitted from light source 2 and reflected by first reflecting area 15 and second reflecting area 17 of reflecting member 3, the decrease in illuminance with distance from light source 2 can be compensated by shortening the optical path length. As a result, the illuminance can be homogenized at the irradiation surface of the irradiation member.

According to the first exemplary embodiment, wavelength transmission unit 5 transmits the radiant light, which has the effect to suppress the production of the inflammatory cytokine and has the wavelength ranging from 566.5 nm to 780 nm. At this point, the wavelength of 780 nm that is the upper limit of the radiant light transmitted through wavelength transmission unit 5 is the upper limit of the visible light (ray). For this reason, while the production of the inflammatory cytokine is suppressed, the thermal influence can be controlled to the minimum level when the region in which the disease should be prevented or the diseased region (including the region that has the effect to treat the diseased region because the region is medically correlated with the diseased region) is irradiated with the radiant light.

In the light irradiation treatment and prevention device of the first exemplary embodiment, the production of the inflammatory cytokine can be suppressed by irradiating the region in which various diseases (such as an inflammation and a rough skin) should be prevented or the diseased region with the radiant light transmitted through wavelength transmission unit 5. As a result, the production of the inflammatory cytokine is suppressed to prevent the inflammatory disease, and the symptom of the disease can be reduced or suppressed.

### Second Exemplary Embodiment

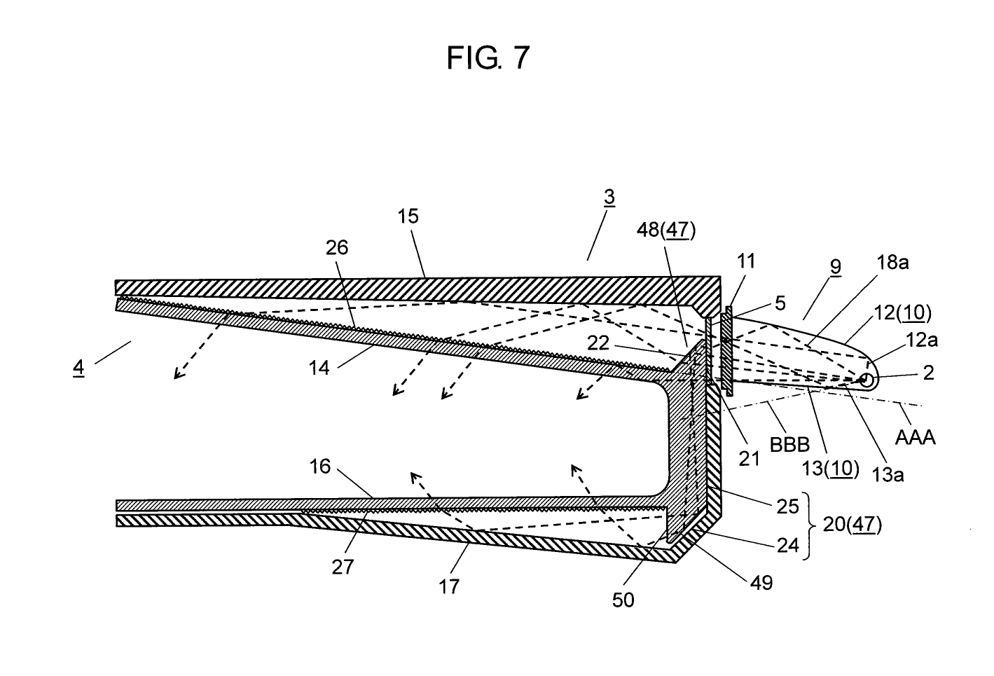

A light irradiation device according to a second exemplary embodiment of the present invention and a light irradiation treatment and prevention device provided therewith will be described below with reference to FIG. 7. Because a basic configuration of the light irradiation treatment and prevention device of the second exemplary embodiment is identical to that of light irradiation treatment and prevention device 1 of the first exemplary embodiment, the description thereof is omitted. The description of the basic configuration of the light irradiation treatment and prevention device is also omitted in the subsequent exemplary embodiment. A constituent different from that of the first exemplary embodiment is designated by a new numeral, and the new constituent will mainly be described below.

FIG. 7 is a cross-sectional view illustrating the light irradiation device of the light irradiation treatment and prevention device of the second exemplary embodiment of the present invention.

As illustrated in FIG. 7, in third reflecting area 47 of reflecting member 3 of the light irradiation device constituting light irradiation treatment and prevention device 1 of the second exemplary embodiment, the functions from reflecting surface 22 of prism 19 of the first exemplary embodiment to reflecting surface 24 of reflecting unit 20 are configured by prism 48 that is continuously and integrally made of a material having the same refractive index. Prism 48 has a substantial parallelogram (including a parallelogram) shape in section. At this point, prism 48 is formed in an end face on the side of light source 2 of substantial U-shape irradiation member 4 while being integral with irradiation member 4.

As illustrated in FIG. 7, like prism 19 of the first exemplary embodiment, prism 48 includes incident surface 21, first reflecting surface 22, second reflecting surface 49, and transmission surface 50. First reflecting surface 22 of prism 48 corresponds to the reflecting surface of prism 19 of the first exemplary embodiment. Second reflecting surface 49 of prism 48 includes a surface that is substantially parallel (including parallel) to reflecting surface 24 of reflecting unit 20 to face reflecting surface 24, and second reflecting surface 49 reflects the radiant light emitted from light source 2 toward second reflecting area 17. Transmission surface 50 of prism 48 includes a surface that is substantially parallel (including parallel) to incident surface 21, and transmission surface 50 transmits the radiant light reflected by the second reflecting surface 49 toward second reflecting area 17 without scattering.

According to the second exemplary embodiment, in the radiant light transmitted through Fresnel lens 11, radiant light 18a, which is transmitted through wavelength transmission unit 5, is incident to third reflecting area 47, and is reflected toward second reflecting area 17 by first reflecting surface 22 of prism 48, is reflected by second reflecting surface 49 of prism 48 like light irradiation treatment and prevention device 1 of the first exemplary embodiment. Then, the reflected radiant light 18a is substantially transmitted through transmission surface 50 without scattering, reflected by second reflecting area 17 of reflecting member 3, and is incident to second irradiation surface 16 of irradiation member 4. Radiant light 18a incident to second irradiation surface 16 is refracted onto the side of light source 2 by prism 27 of second irradiation surface 16 so as not to be leaked to the outside (the opposite side to light source 2), and the irradiation object (for example, the hand) opposed to second irradiation surface 16 is irradiated with radiant light 18a.

According to the second exemplary embodiment, first reflecting surface 22 and second reflecting surface 49 are continuously and integrally molded using a translucent member having the same refractive index, which allows the reflection efficiency to be enhanced in second reflecting surface 49. As a result, a quantity of electric power applied to light source 2 can be reduced in the case that the same light quantity as the first exemplary embodiment is implemented. On the other hand, a quantity of light with which the irradiation object is irradiated can be increased in the case that the same power quantity as the first exemplary embodiment is applied to light source 2.

### Third Exemplary Embodiment

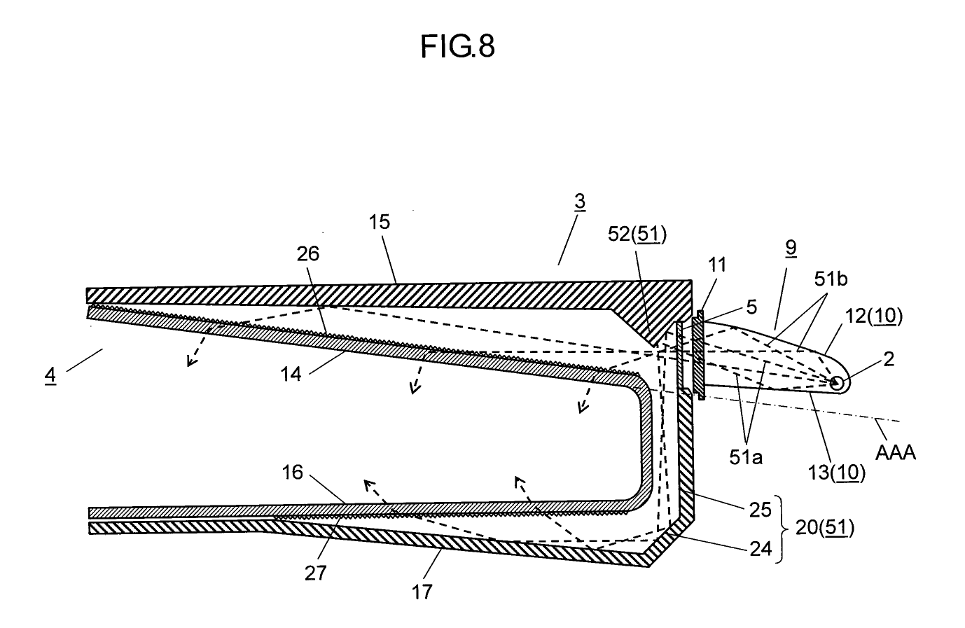

A light irradiation device according to a third exemplary embodiment of the present invention and a light irradiation treatment and prevention device provided therewith will be described below with reference to FIG. 8.

FIG. 8 is a cross-sectional view illustrating the light irradiation device of the light irradiation treatment and prevention device of the third exemplary embodiment of the present invention.

As illustrated in FIG. 8, in the light irradiation device constituting light irradiation treatment and prevention device 1 of the third exemplary embodiment, instead of prism 19 constituting third reflecting area 51 of reflecting member 3 of the first exemplary embodiment, for example, reflecting projection 52 having a right-angled triangular shape in section is provided as third reflecting area 51 in an end face on the side of light source 2 of first reflecting area 15 while projecting toward first irradiation surface 14 of irradiation member 4.

As illustrated in FIG. 8, reflecting projection 52 is disposed such that the surface opposed to wavelength transmission unit 5 has the same inclination (45 degrees angle) as reflecting surface 22 of prism 19 of the first exemplary embodiment. At this point, reflecting projection 52 is provided while projecting toward the side of irradiation member 4 up to the extent that about a half of radiant light 51a, which is emitted from light source 2 toward the side of first reflecting area 15 with respect to wavelength transmission unit 5, is blocked. Therefore, reflecting projection 52 reflects about a half of radiant light 51a, which is emitted from light source 2 toward the side of first reflecting area 15, toward second reflecting area 17 of reflecting member 3 in the radiant light transmitted through wavelength transmission unit 5.

According to the third exemplary embodiment, in the radiant light that is emitted from light source 2 toward first reflecting area 15, radiant light 51a in which the optical path to first reflecting area 15 is blocked by reflecting projection 52 of third reflecting area 51 is guided to second reflecting area 17, and the irradiation object is irradiated with radiant light 51a from second irradiation surface 16.

That is, radiant light 51a that is part of the radiant light is reflected toward reflecting surface 24 of reflecting unit 20 by reflecting projection 52 of third reflecting area 51, and reflected toward second reflecting area 17 by reflecting surface 24. Radiant light 51a reflected toward reflecting member 3 by second reflecting area 17 is transmitted through second irradiation surface 16 of irradiation member 4, and the irradiation object is irradiated with radiant light 51a.

On the other hand, in the radiant light emitted from light source 2 toward first reflecting area 15, remaining radiant light 51b that is not blocked by reflecting projection 52 is directly transmitted through first irradiation surface 14 of irradiation member 4, and the irradiation object is irradiated with radiant light 51b. Therefore, the pieces of radiant light that are emitted from one light source 2 to travel toward second reflecting area 17 through first reflecting area 15 and third reflecting area 51 are transmitted through first irradiation surface 14 and second irradiation surface 16 corresponding to each reflecting area, and the irradiation object can simultaneously be irradiated with the pieces of radiant light.

The light irradiation treatment and prevention device of the present invention is not limited to the above exemplary embodiments, but various changes can be made without departing from the scope of the present invention.

For example, in light irradiation treatment and prevention device 1 of the exemplary embodiments, the hand is irradiated by way of example. However, light irradiation treatment and prevention device 1 is not limited to the hand. For example, the region in which the production of the inflammatory cytokine should be suppressed of another living body or the diseased region of another living body may be irradiated. As to the region of the living body, any place such as a shoulder, a waist, a leg, and a total body may be irradiated. In addition to the case that the human is irradiated, specific regions of living bodies such as an animal except the human may be irradiated with the radiant light for the purpose of the treatment. In this case, light irradiation treatment and prevention device 1 is not limited to the structures of the exemplary embodiments, but light irradiation treatment and prevention device 1 can properly be changed to the structure suitable to the specific region of the living body to be irradiated.

In light irradiation treatment and prevention devices 1 of the exemplary embodiments, by way of example, the region of the living body in which production of the inflammatory cytokine should be suppressed or the diseased region of the living body is irradiated for the purpose of the treatment or the prevention. However, light irradiation treatment and prevention devices 1 of the exemplary embodiments are not limited to the treatment or the prevention. For example, light irradiation treatment and prevention device 1 may be used as a lighting device, particularly a surface lighting device, such as general lighting, light for a signboard, lighting for store shelves of the automatic vending machine, and a backlight of the liquid crystal display, in which the irradiated bodies uniformly aligned or arrayed need to be efficiently and homogeneously illuminated. Therefore, the irradiation object can homogeneously be irradiated with the irradiation light output from the irradiation member.

By way of example, light irradiation treatment and prevention devices 1 of the exemplary embodiments include wavelength transmission units 5 each of which transmits the radiant light having the specific wavelength range. However, light irradiation treatment and prevention device 1 is not limited to the exemplary embodiments. For example, wavelength transmission unit 5 may not be provided in the case that light irradiation treatment and prevention device 1 is used as the surface lighting device. Light irradiation treatment and prevention device 1 may include wavelength transmission unit 5 that transmits the radiant lights having different wavelengths (frequencies) according to the irradiation object.

In light irradiation treatment and prevention devices 1 of the exemplary embodiments, by way of example, the bandpass filter is used as wavelength transmission units 5. However, light irradiation treatment and prevention device 1 is not limited to the exemplary embodiments. For example, wavelength transmission unit 5 that selectively transmits the wavelength of the specific range of the incident light may be used by combining a short-pass filter (a long-wavelength cutoff filter) and a long-pass filter (a short-wavelength cutoff filter).

In light irradiation treatment and prevention devices 1 of the exemplary embodiments, by way of example, one or two cylindrical flash discharge tubes are used as the light source. However, light irradiation treatment and prevention device 1 is not limited to the exemplary embodiments. For example, at least two flash discharge tubes arrayed in series in an axial direction or a plurality of linearly-arrayed LEDs may be used as the light source. A light source that corresponds to the irradiation member extended in a width direction may be used.

In light irradiation treatment and prevention devices 1 of the exemplary embodiments, by way of example, irradiation member 4 includes the planar irradiation surface. However, light irradiation treatment and prevention device 1 is not limited to the exemplary embodiments. For example, the irradiation surface of the irradiation member may be formed into an arc shape that is rounded in a depth direction (the direction from the opening on the front end side of irradiation member 4 toward the side of light source 2) or a semicircular shape. In this case, preferably the light source is disposed on the irradiation object side with respect to the tangent of the end portion on the light source side in the irradiation surface of the irradiation member.

In light irradiation treatment and prevention devices 1 of the exemplary embodiments, by way of example, first irradiation surface 14 and second irradiation surface 16 of irradiation member 4 do not have the diffusion property. However, light irradiation treatment and prevention device 1 is not limited to the exemplary embodiments. For example, a diffusion panel having the diffusion property may be used in the irradiation surface of the irradiation member.

In light irradiation treatment and prevention devices 1 of the exemplary embodiments, by way of example, the two irradiation surfaces are provided as first irradiation surface 14 and second irradiation surface 16 of irradiation member 4 with respect to one light source 2. However, light irradiation treatment and prevention device 1 is not limited to the exemplary embodiments. For example, at least three irradiation surfaces may be provided with respect to one light source 2. In this case, the third reflecting area of the reflecting member is provided according to the increased irradiation surface, and the radiant light may be guided to each irradiation surface of the irradiation member. Therefore, not only the two surfaces such as the surface and the backside of the hand, but also the irradiation object having a plurality of surfaces such as a lateral surfaces of the hand can simultaneously be irradiated with the radiant light emitted from the one light source. As a result, the high-efficiency light irradiation device that can irradiate the irradiation object with radiant light in a short time and the light irradiation treatment and prevention device 1 provided therewith can be configured.

In light irradiation treatment and prevention devices 1 of the exemplary embodiments, by way of example, the third reflecting area of the reflecting member blocks about a half of the radiant light emitted onto the irradiation member side or about a half of the radiant light emitted onto the first reflecting area side of the reflecting member in the radiant light emitted from the light source toward the first reflecting area. However, light irradiation treatment and prevention device 1 is not limited to the exemplary embodiments. For example, a relationship (a ratio) between the irradiation quantity output from the first irradiation surface of the irradiation member and the irradiation quantity output from the second irradiation surface may be changed. In this case, the irradiation quantity that is blocked on the irradiation member side or the first reflecting area side of the reflecting member may arbitrarily be changed by the third reflecting area of the reflecting member in the radiant light that is emitted from the light source toward the first reflecting area.

As described above, a light irradiation device of the present invention includes: a light source that emits radiant light to an irradiation object; a reflecting member that reflects the radiant light emitted from the light source toward the irradiation object; and an irradiation member that includes at least two irradiation surfaces for transmitting the radiant light reflected from the reflecting member and irradiating the irradiation object with the radiant light. The light source emits the radiant light from one end side of the irradiation surfaces of the irradiation member, and the reflecting member includes: a first reflecting area that reflects the radiant light emitted from the light source toward a first irradiation surface of the irradiation member; a second reflecting area that reflects the radiant light emitted from the light source and indirectly reaches the second reflecting area toward a second irradiation surface of the irradiation member; and a third reflecting area that guides the radiant light to the second reflecting area.

According to the configuration, the irradiation member includes the plurality of irradiation surfaces. Part of the radiant light emitted from the light source toward each irradiation surface of the irradiation member is reflected by the third reflecting area, indirectly guided to the second reflecting area and reflected by one of the first reflecting area and the second reflecting area of the reflecting member. Therefore, the radiant light reflected by each reflecting area is incident to and transmitted through the irradiation surface corresponding to the irradiation member, and the irradiation object can be irradiated with the radiant light. As a result, using the one light source, the irradiation object having the surface and the backside can simultaneously be irradiated with the pieces of radiant light transmitted through the plurality of irradiation surfaces.

In the light irradiation device of the present invention, the first reflecting area and the second reflecting area of the reflecting member are disposed to face each other, and the third reflecting area partially blocks an optical path of the radiant light emitted from the light source toward the first reflecting area, and guides the radiant light to the second reflecting area.

According to the configuration, the irradiation object is irradiated through the first irradiation surface with the radiant light emitted from the light source toward the first reflecting area. On the other hand, part of the radiant light in which the optical path to the first reflecting area is blocked by the third reflecting area is guided to the second reflecting area, and the irradiation object is irradiated through the second irradiation surface with the radiant light. Therefore, the irradiation object can simultaneously be irradiated from each corresponding irradiation surface.

In the light irradiation device of the present invention, the irradiation member is formed to refract the radiant light transmitted through the irradiation surface of the irradiation member at an angle smaller than an incident angle to the irradiation surface.

According to the configuration, the light source side of the irradiation surface is irradiated with the radiant light output from the irradiation member. Therefore, the light irradiation device, which irradiates the irradiation object such that the radiant light output from the irradiation member does not spread to the outside, can be configured.

A light irradiation treatment and prevention device of the present invention performs treatment or prevention by irradiating a specific region of an irradiation object with radiant light output from the light irradiation device, the light irradiation treatment and prevention device includes a wavelength transmission unit that is disposed to an optical path of the radiant light emitted from a light source and output from an irradiation member, and transmits the radiant light having wavelengths ranging from 566.5 nm to 780 nm inclusive, wherein the specific region of the irradiation object is irradiated with the radiant light transmitted through the wavelength transmission unit.

According to the configuration, the irradiation member includes the plurality of irradiation surfaces. Part of the radiant light emitted from the light source toward each irradiation surface of the irradiation member is reflected by the third reflecting unit, indirectly guided to the second reflecting unit, and reflected by one of the first reflecting unit and the second reflecting unit of the reflecting member, which allows the irradiation object to be irradiated from the irradiation surface of the irradiation member corresponding to each reflecting unit. As a result, using the one light source, the irradiation object having the surface and the backside can simultaneously be irradiated by emitting the radiant light to the plurality of irradiation surfaces.

According to the configuration, the production of the inflammatory cytokine can be suppressed by irradiating the region in which various diseases (such as the inflammation and the rough skin) should be prevented or the diseased region with the radiant light transmitted through wavelength transmission unit. As a result, the production of the inflammatory cytokine is suppressed to prevent the inflammatory disease, and the symptom of the disease can be reduced or suppressed.

Specifically, wavelength transmission unit of the above configuration transmits the radiant light, which has the effect to suppress the production of the inflammatory cytokine and has the wavelength ranging from 566.5 nm to 780 nm which was found by the present inventor. At this point, the wavelength of 780 nm that is the upper limit of the radiant light transmitted through wavelength transmission unit is the upper limit of the visible light (ray). For this reason, while the production of the inflammatory cytokine is suppressed, the thermal influence can be controlled to the minimum level when the region in which the disease should be prevented or the diseased region (including the region that has the effect to treat the diseased region because the region is medically correlated with the diseased region) is irradiated with the radiant light.

### INDUSTRIAL APPLICABILITY

The present invention can be applied to the light irradiation device and the light irradiation treatment and prevention device, which need to homogeneously and simultaneously irradiate the plurality of irradiation surface of the irradiation member with the radiant light emitted from the one light source.

### REFERENCE MARKS IN THE DRAWINGS

1 treatment and prevention device
2 light source (discharge tube)
3 reflecting member
4 irradiation member
5 wavelength transmission unit
6 emission controller
7 power-supply unit
8 device body
9 light source unit
10 reflector
11 Fresnel lens
12 first reflecting unit
12a, 13a, 13b, 18a, 51a, 51b radiant light
12R first reflecting plate
13 second reflecting unit
13R second reflecting plate
14 first irradiation surface
15 first reflecting area
16 second irradiation surface
17 second reflecting area
18, 47, 51 third reflecting area
19 prism
20 reflecting unit
21 incident surface
22, 24 reflecting surface (first reflecting surface)
23, 50 transmission surface
25 guide surface
26 prism
27 prism
28 emission operation controller
29 operation display unit
30 irradiation state displaying LED
31 warning LED
32 waiting time display
33 electric accumulator
34 charging circuit
35 power unit
36 power switch
37 plug
38 power cable
39 opening
40 placement unit
41 leaky light preventing unit
42 cooling unit
43 handle
44 guide
45 automatic starting sensor
46 exhaust port
48 prism
49 second reflecting surface
52 reflecting projection

1. A light irradiation device comprising:
a light source that emits radiant light to an irradiation object;
a reflecting member that reflects the radiant light emitted from the light source toward the irradiation object; and
an irradiation member that includes at least two irradiation surfaces for transmitting the radiant light reflected from the reflecting member and irradiating the irradiation object with the radiant light,
wherein the light source emits the radiant light from one end side of the irradiation surfaces of the irradiation member, and
the reflecting member includes:
a first reflecting area that reflects the radiant light emitted from the light source toward a first irradiation surface of the irradiation member;
a second reflecting area that reflects the radiant light emitted from the light source and indirectly reaches the second reflecting area toward a second irradiation surface of the irradiation member; and
a third reflecting area that guides the radiant light to the second reflecting area.

2. The light irradiation device according to claim 1, wherein the first reflecting area and the second reflecting area of the reflecting member are disposed to face each other, and the third reflecting area partially blocks an optical path of the radiant light emitted from the light source toward the first reflecting area, and guides the radiant light to the second reflecting area.

3. The light irradiation device according to claim 1, wherein the irradiation member is formed to refract the radiant light transmitted through the irradiation surface of the irradiation member at an angle smaller than an incident angle to the irradiation surface.

4. A light irradiation treatment and prevention device that performs treatment or prevention by irradiating a specific region of an irradiation object with radiant light output from the light irradiation device according to claim 1,
the light irradiation treatment and prevention device comprising a wavelength transmission unit that is disposed to an optical path of the radiant light emitted from a light source and output from an irradiation member, and transmits the radiant light having wavelengths ranging from 566.5 nm to 780 nm inclusive,
wherein the specific region of the irradiation object is irradiated with the radiant light transmitted through the wavelength transmission unit.

5. The light irradiation device according to claim 2, wherein the irradiation member is formed to refract the radiant light transmitted through the irradiation surface of the irradiation member at an angle smaller than an incident angle to the irradiation surface.
